(19) United States
(12) Patent Application Publication
(10) Pub. No.: US 2008/0287936 A1
(43) Pub. Date: Nov. 20, 2008

(54) Telescope with Integrated Optical Filter

(76) Inventors: Douglas G. Stinson, Fremont, CA (US); Kester Nahen, Heidelberg (DE)

Correspondence Address: AMS RESEARCH CORPORATION, 10700 BREN ROAD WEST, MINNETONKA, MN, 55343, US

(21) Appl. No.: 12/122,804

(22) Filed: May 19, 2008

Related U.S. Application Data
(60) Provisional application No. 60/938,738, filed on May 18, 2007.

(51) Int. Cl. A61B 18/20

(52) U.S. Cl. 606/13

(57) ABSTRACT

A medical laser system having a telescope and a laser unit, wherein the laser unit includes an optical fiber for performing medical surgical procedures, and a telescope designed to view a target area during the medical procedure and to illuminate the target area. The telescope includes a tubular case, an optical train, an integrated optical filter, and an optical fiber to provide illumination. The telescope can be connected to a camera to allow for viewing the target area on a monitor. Alternatively, the telescope can end in an eyepiece. The integrated optical filter blocks a selected laser light wavelength such that the laser light does not overexpose or damage the camera. The integrated optical filter can include an integrated flat filter or integrated coated lens.

### PRIORITY CLAIM

The present application claims priority to U.S. Provisional Application Ser. No. 60/938,738 filed May 18, 2007, and entitled, “TELESCOPE WITH INTEGRATED OPTICAL FILTER”, which is herein incorporated by reference in its entirety.

### FIELD OF THE DISCLOSURE

The invention relates generally to the field of medical laser systems utilizing a telescope to position an optical fiber tip to treat targeted tissue during a medical procedure. More specifically, the present invention is directed to a telescope including an integrated optical filter to block desired laser light wavelengths so as to avoid damage/overexposure to cameras used to observe positioning of the optical fiber tip.

### BACKGROUND OF THE INVENTION

Medical lasers have been used in treatment procedures involving various practice areas including, for example, urology, neurology, otorhinolaryngology, general anesthetic opthalmology, dentistry, gastroenterology, cardiology, gynecology, and thoracic and orthopedic procedures. Generally, these procedures require precisely controlled delivery laser energy, and often the area to which the energy is to be delivered is located deep within the body, for example, at the prostate or at the fallopian tubes. Due to the location of the target tissue deep within the body, the medical procedure generally requires use of a flexible and maneuverable optical fiber. Depending upon the requirements for a light source, a variety of light sources can be used in conjunction with the optical fiber including, for example, pulsed lasers, diode lasers, and neodymium lasers. Representative lasers used in medical treatment procedures include Ho:YAG lasers and Nd:YAG lasers.

Often, an instrument, such as a laparascope, bronchoscope, gastroscope, cytoscope, endoscope or similar instrument (generically, a “telescope”) is utilized during the treatment of body tissue with laser light. This telescope instrument generally comprises a flexible tube, a light delivery system such as an optical fiber system for illuminating the tissue or organ under examination and a lens system transmitting the image to the viewer. The telescope generally has a viewing apparatus eyepiece at one end and an objective lens at the other end. The lens is often a wide-angle lens. The light source of the system is commonly an external light source and an optical fiber directs the light source into the orifice or body cavity to illuminate the subject tissue or organ so that it is clearly visible.

The various telescopes that are used for visual examination of internal organs and tissues, and body cavities, are generally of similar structure, but of different size. An optical relay carries the image to the viewing apparatus, surrounding optical fibers transmit light to the object, and the viewing apparatus allows for viewing of the subject object, in this instance, tissue. Generally, the viewing apparatus includes a camera attached to the telescope instrument so that the image generated by the camera can be used to direct activity during the medical procedure. The telescopes can be stereo telescopes, such that the image that is provided is a three-dimensional image, which facilitates more accurate maneuvering in the body cavity or orifice. Further, the telescope can include an additional channel that can facilitate entry of medical instruments, including a medical laser optical fiber with a surgical probe. Hence, a medical laser can be used along with a telescope, where the laser light is used to accomplish the medical procedure, and the telescope is used to view the target area to guide the surgeon during the medical procedure and/or to illuminate the required area.

When a high intensity laser light is used in the medical laser procedure, the laser light can overexpose and damage the camera that is affixed to the telescope to observe and guide the medical procedure. Therefore, a separate filter is used to block the laser light wavelength emitted by the particular laser used for the medical procedure while allowing other wavelengths of light to pass through the filter, to the greatest extent possible. The separate filter is placed between a proximal end of the telescope and the camera. The separate filter allows for easy replacement, however, it is also easy to lose or misplace the separate filter. Because the separate filter is an additional piece of equipment, the separate filter must be installed in the proper location prior to use of the camera, telescope and medical procedure laser. Further, a method of identifying the proper separate filter to be installed, with respect to the wavelength of laser light being used, and ensuring that the needed separate filter is available and properly installed is required. Unfortunately, the separate filter introduces two additional optical surfaces that can fog due to the moisture present in the operating environment, thus obscuring viewing of the tissue and potentially delaying the medical procedure.

Hence, there remains a need for an optical filter that blocks the targeted light wavelengths and allows for passage of the other wavelengths. Furthermore, a suitable filter must be readily available and identifiable, and should not introduce additional problems into the telescope system by adding additional optical surfaces that may fog during the medical procedure.

### SUMMARY OF THE INVENTION

The present invention comprises a medical laser system with an optical fiber for delivery of laser energy to target tissue and a telescope designed to view the target tissue during the medical procedure. The telescope system includes a tubular case enclosing an optical system having an integrated optical filter for removing a selected laser wavelength from an image being transmitted to a camera for viewing the target tissue on an associated monitor. Generally, the integrated optical filter blocks the selected laser light wavelength to prevent overexposure and related damage to the camera. In some embodiments, the integrated optical filter can comprise a flat filter. Alternatively, the integrated optical filter can comprise a coated lens. In some embodiments, the telescope can comprise a viewing apparatus having an external color selected to correspond to a laser wavelength filtered by the integrated optical filter such as, for example, a green viewing apparatus for filter 532 nm laser wavelength light (green light).

In another aspect of the present invention, a method for protecting a camera from overexposure and related damage during a medical laser procedure can comprise providing a medical telescope having a flexible tube with an optical system mounted therein, the optical system including an integrated optical filter. The method can further comprise transmitting a target image through the optical system. Finally, the method can comprise filtering a selected laser wavelength with the integrated optical filter prior to transmitting the target image to a camera. In some embodiments, the method can further comprise coloring a viewing apparatus on the telescope with an exterior color indicative of the selected laser wavelength filtered by the integrated optical filter.

Although specific examples have been illustrated and described herein, it will be appreciated by those of ordinary skill in the art that any arrangement calculated to achieve the same purpose could be substituted for the specific examples shown.

### BRIEF DESCRIPTION OF THE DRAWINGS

These as well as other objects and advantages of this invention will be more completely understood and appreciated by referring to the following more detailed description of the presently preferred exemplary embodiments of the invention in conjunction with the accompanying drawings, of which:

FIG. 1 is a schematic of a laser system with an optical fiber attached to the laser unit.

FIG. 2 is a side, partially hidden view of a monoscopic telescope system having a flat optical filter according to an embodiment of the present invention.

FIG. 3 is a side, partially hidden view of a monoscopic telescope system having a coated lens according to an embodiment of the present invention.

FIG. 4 is a side, partially hidden view of a stereoscopic telescope system according to an embodiment of the present invention.

FIG. 5 is a flow chart illustrating a method of protecting a camera from overexposure to laser wavelengths according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives.

### DETAILED DESCRIPTION OF EMBODIMENTS OF THE INVENTION

The present invention includes a medical laser system having a telescope and a laser unit, wherein the laser unit includes an optical fiber for performing medical surgical procedures, and a telescope designed to illuminated and view a target area during the medical procedure. The telescope includes a tubular case, an optical train, an integrated optical filter, and an optical fiber to provide illumination. The telescope can be connected to a camera to allow for viewing the target area on a monitor. Alternatively, the telescope can include an eyepiece. The integrated optical filter blocks a selected laser light wavelength such that the laser light does not overexpose or damage the camera. The integrated optical filter can include a flat filter or coated lens. In one preferred embodiment, the telescope of the present invention is utilized with a Greenlight HPS system manufactured by American Medical Systems of Minnetonka, Minn. and as described in U.S. Pat. Nos. 6,554,824 and 6,986,764, which are herein incorporated by reference.

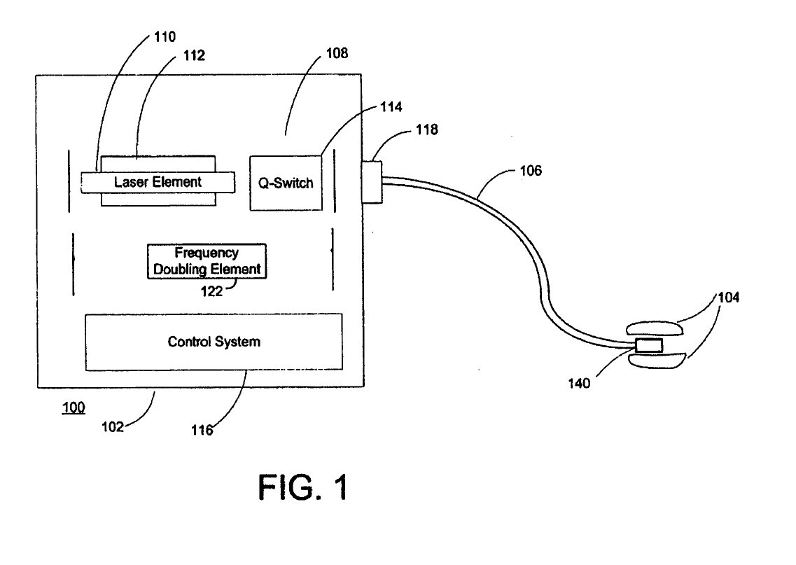

Referring to FIG. 1, there is depicted a block diagram showing an exemplary laser system 100 which can be employed for implementing the present invention. Laser system 100 includes a solid-state laser unit 102, which is used to generate laser light for delivery through optical fiber 106 to target tissue 104. Laser unit 102 is capable of being operated in a pulsed mode or continuous wave.

Laser unit 102 more specifically comprises a laser element assembly 110, pump source 112, and frequency doubling crystal 122. In the preferred-embodiment, laser element 110 outputs 1064 nm light which is focused into frequency doubling crystal 122 to create 532 nm light. According to one implementation, laser element assembly 110 may be neodymium doped YAG (Nd:YAG)crystal, which emits light having a wavelength of 1064 nm (infrared light) when excited by pump source 112. Laser element 110 may alternatively be fabricated from any suitable material wherein transition and lanthanide metal ions are disposed within a crystalline host (such as YAG, Lithium Yttrium Fluoride, Sapphire, Alexandrite, Spinel, Yttrium Orthoaluminate, Potassium Gadolinium Tungstate, Yttrium Orthovandate, or Lanthahum Scandium Borate). Laser element 110 is positioned proximal to pump source 112 and may be arranged in parallel relation therewith, although other geometries and configurations may be employed.

Pump source 112 can be any device or apparatus operable to excite laser element assembly 110. Non-limiting examples of devices which can be used as pump source 112, include: arc lamps, flashlamps, and laser diodes.

A Q-switch 114 disposed within laser unit 102 can be operated in a repetitive mode to cause a train of micropulses to be generated by laser unit 102. Typically the micropulses are less than 1 microsecond in duration separated by about 40 microseconds, creating a quasi-continuous wave train. Q-switch 114 is preferably of the acousto-optic type, but can alternatively comprise a mechanical device such as a rotating prism or aperture, an electro-optical device, or a saturable absorber.

Laser unit 102 is provided with a control system 116 for controlling and operating laser unit 102. Control system 116 will typically include a control processor which receives input from user controls (including but not limited to a beam on/off control, a beam power control, and a pulse duration control) and processes the input to accordingly generate output signals for adjusting characteristics of the output beam to match the user inputted values or conditions. With respect to pulse duration adjustment, control system 116 applies an output signal to a power supply (not shown) driving pump source 112 which modulates the energy supplied thereto, in turn controlling the pulse duration of the output beam.

Although FIG. 1 shows an internal frequency doubled laser, it is only by way of example. The infrared light can be internally or externally frequency doubled using non-linear crystals such as KTP, Lithium Triborate (LBO), or Beta Barium Borate (BBO) to produce 532 nm light. The frequency doubled, shorter wavelength light is better absorbed by the hemoglobin and char tissue, and promotes more efficient tissue ablation. Finally, the green light leaves only a thin char layer with little pre and post operative bleeding.

Laser unit 102 further includes an output port 118 couplable to optical fiber 106. Output port 118 directs the light generated by laser unit 102 into optical fiber 106 for delivery to tissue 104. While a bare fiber may be utilized for certain procedures, optical fiber 106 preferably terminates in a tip 140 having optical elements for shaping and/or orienting the beam emitted by optical fiber 106 so as to optimize the tissue ablation process, for example a side-firing fiber. At times it is necessary to physically move the laser unit 100 between different treatment locations

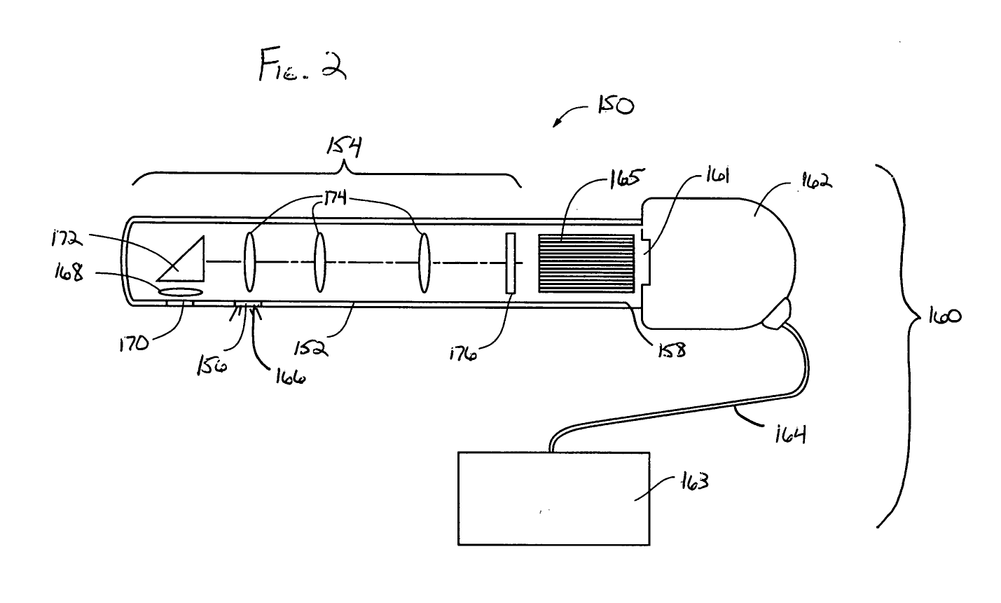

As illustrated in FIG. 2, a telescope system 150 for use with laser unit 100 generally comprises a tubular case 152 enclosing an optical system 154. In order to facilitate its use in medical purposes, flexible case 152 generally has a long and thin configuration providing for maneuvering within the human body. The telescope system 150 also includes a light delivery system 156, such as, for example, an optical fiber 158 to illuminate the target tissue or organ under examination. Optical system 154 allows the telescope system 150 to transmit the image of the target tissue to a viewing mechanism 160 such as, for example, an eyepiece 161 for direct viewing, or more preferably, a camera 162 connected to an associated monitor 163 by a transmitting cable 164. Optical system 154 includes an optical fiber bundle 165 for transmitting the image to the viewing mechanism 160.

Optical fiber 158 in the telescope system 150 transmits illuminating light through an aperture 166 in the tubular case 152 to the target tissue such that the target tissue is illuminated and visible. The image of the target tissue is captured by an objective lens 168 through a viewing window 170 in the tubular case 152. The image of the target tissue is transmitted by way of a mirror 172 and a series of lenses 174 comprising the optical system 154. The image is then carried to the viewing mechanism 160 using optical fiber bundle 165 for subsequent viewing with camera 162 and associated monitor 163. Alternatively, the optical fiber bundle 165 can transmit the captured image to eyepiece 161 for direct viewing by a medical professional. As depicted, telescope system 150 is configured to provide a monocular view of the target tissue. When telescope system 150 is configured for a monocular view, the image transmitted to the viewing mechanism 160 does not provide for depth perception and, therefore, surgical procedures must be practiced and learned without the benefit of a three-dimensional image.

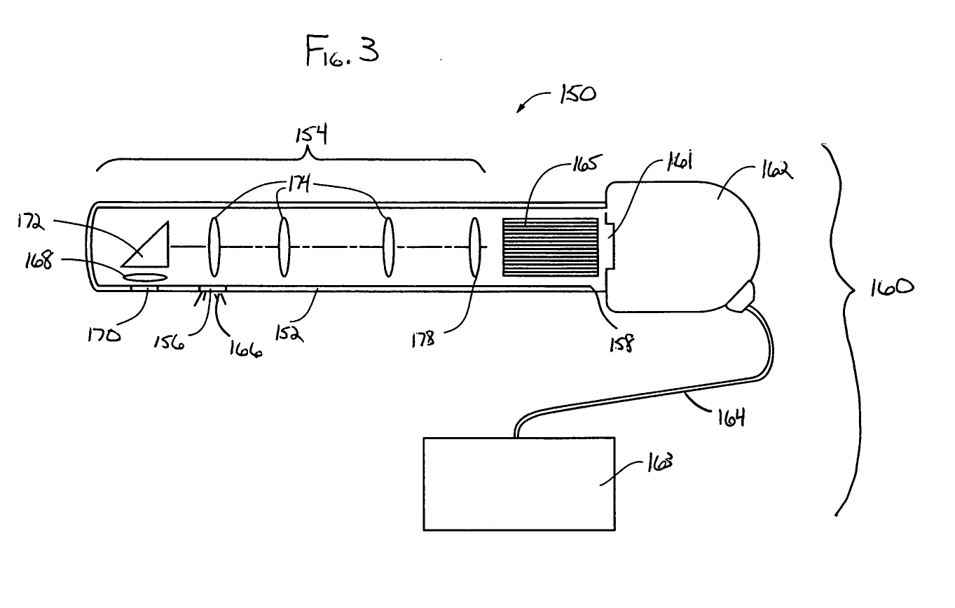

As high intensity light, for example, laser light utilized to perform laser medical procedures, can overexpose and damage the camera 162, telescope system 150 of the present invention includes an integrated optical filter 176 contained as part of the optical system 154. Integrated optical filter 176 blocks a selected laser light wavelength from passing through the optical fiber bundle 165 to the camera 162 while still allowing for other wavelengths of light to successfully reach the camera 162 for image display. In this manner, the desired image can be transmitted for viewing without risking overexposure or damage to the camera 162. In a preferred embodiment, the integrated optical filter 176 is a flat configuration so as to simplify a coating design through limiting the angles of incidence over which the coating must meet filtering requirements. In an alternative embodiment illustrated in FIG. 3, optical system 154 can include one or more integrated coated lenses 178 having a specified coating to reflect and/or absorb light at a selected laser wavelength, generally a laser wavelength that is desirable to prevent from reaching the camera 162. The integrated optical filter 176 or integrated coated lens 178 is especially useful when the laser emits light in the region of the spectrum where the camera 162 is sensitive, typically, the visible region. As noted above, lasers that emit 532 nm wavelengths are used in a variety of surgical procedures involving tissue ablation and vaporization. As such, integrated optical filter 176 and integrated coated lens 178 are frequently configured to block, reflect or absorb 532 nm light.

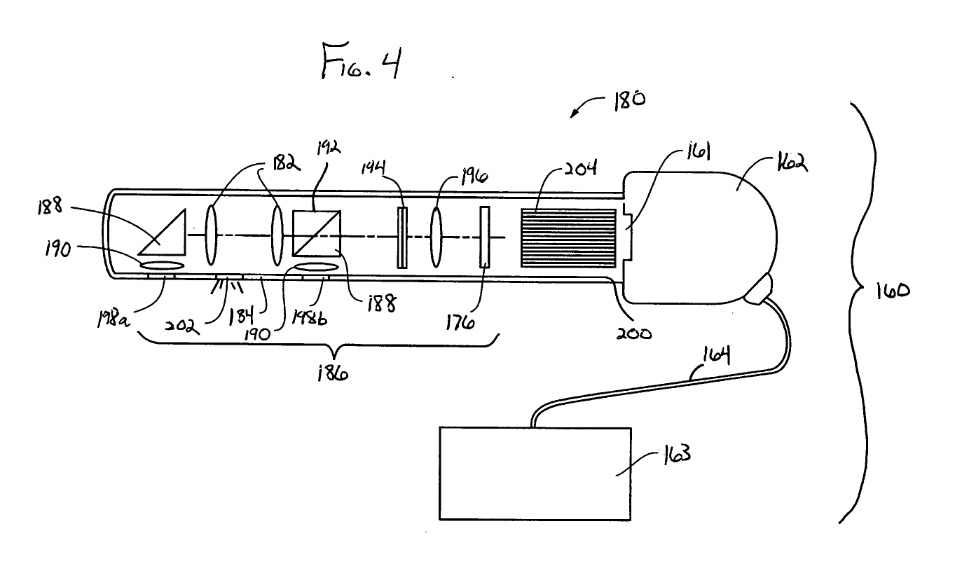

Referring to FIG. 4, a telescope system 180 can create a stereoscopic image through the combination of two dimensional images, for example, a left image and a right image. With telescope system 180, lenses 182 are positioned within a tubular case 184 to process the left and right optical images. The images are transmitted through the tubular case 184 by way of an optical relay 186 that can include mirrors 188, imaging lenses 190, a polarizing beam splitter 192, an optical switch 194 and a focusing lens 196. Instead of a single window to view the target area as utilized with the monoscopic telescope system 150, the stereoscopic system 180 utilizes a pair of windows 198a, 198b. An illuminating optical fiber 200 provides a source of light to illuminate the target area through a light window 202. The image of the target area is transmitted by way of an optical fiber bundle 204 to the camera 162 to be viewed on associated monitor 163. Again, optical relay 186 can include integrated optical filter 176 or alternatively, one or more integrated coated lenses 178 to remove, reflect or absorb light at a selected laser wavelength so as to prevent the specified wavelength form reaching and damaging the camera 162.

The optical density of the integrated optical filter 176 and coated lens 178 at the desired blocking wavelength can be adjusted according to the sensitivity of the camera 162 and the intensity of the laser unit 102. Preferably, the optical density is in the range of about 3 to 7. In a preferred embodiment, the optical density is about 5.

With monoscopic telescope 150 or stereoscopic telescope 180 that include the integrated optical filter 176 or integrated coated lens 178, the viewing mechanism 160 such as, for example, eyepiece 161 for direct viewing, or the camera 162 and associated monitor 163 can be colored to identify that the telescoped 150, 180 contains the integrated optical filter 176 or coated lens 178. Further, the color of the viewing mechanism 160 can be chosen to be indicative of the wavelength of light that is blocked. For example, if monoscopic telescope 150 includes integrated filter 176 configured to block 532 nm laser wavelength light (green light), the monoscopic telescope 150 can include a green viewing mechanism 160. With the viewing mechanism 160 colored to correspond with the filtering/blocking/absorbing characteristics of the integrated optical filter 176 or coated lens 178, it is a simple matter to match the appropriate telescope with the wavelength of laser light that requires blocking to avoid damage to camera 162. In addition, the integrated nature of integrated optical filter 176 or coated lens 178 means that there is never a need for a medical professional to locate and install a stand alone filter.

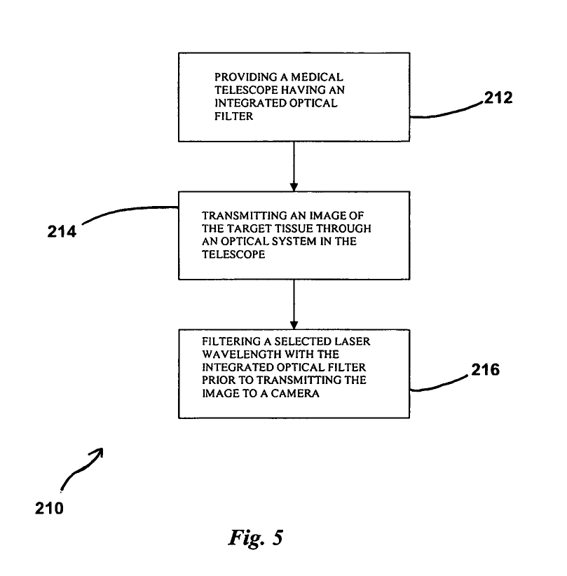

A representative method 210 of preventing overexposure of an image of target tissue for display by a camera during a medical laser procedure is illustrated schematically in FIG. 5. Generally, a first step 212 comprises providing a medical telescope 150 or 180 having a flexible tube with an optical system including an integrated optical filter. The integrated optical filter can comprise an integrated optical filter 176 or coated lens 178. A second step 214 comprises transmit an image of the target tissue though the optical system. A third step 216 comprises filtering a selected laser wavelength with the integrated optical filter prior to transmitting the target image to a camera. In some embodiments, first step 212 can further comprise coloring a viewing apparatus on the telescope with an exterior color indicative of the selected laser wavelength filtered by the integrated optical filter.

Although specific examples have been illustrated and described herein, it will be appreciated by those of ordinary skill in the art that any arrangement calculated to achieve the same purpose could be substituted for the specific examples shown. This application is intended to cover adaptations or variations of the present subject matter. Therefore, it is intended that the invention be defined by the attached claims and their legal equivalents.

We claim:

1. A medical laser system comprising:
a medical laser unit having an optical fiber;
a telescope system having a flexible tubular case enclosing an optical system and a light delivery system, the optical system transmitting a target image to a camera, wherein the optical system includes an integrated optical filter positioned within the flexible tubular case to remove an identified laser wavelength from the target image prior to reaching the viewer.

2. The medical laser system of claim 1, wherein the integrated optical filter comprises a flat filter designed to block the identified laser wavelength.

3. The medical laser system of claim 1, wherein the integrated optical filter comprises a lens having an anti-reflective coating, wherein the anti-reflective coating is designed to block the identified laser wavelength.

4. The medical laser system of claim 1, wherein the integrated optical fiber prevents overexposure of the target image captured by the camera.

6. The medical laser system of claim 1, wherein the integrated optical filter has an optical density from about 3 to about 7.

7. The medical laser system of claim 6, wherein the optical density is about 5.

8. The medical laser system of claim 4, wherein the telescope system further comprises a colored viewing apparatus, wherein a color of the colored viewing apparatus is indicative of the identified laser wavelength filtered by the integrated optical filter.

9. A medical telescope system, comprising:
a flexible tubular case enclosing an optical system and a light delivery system that delivers laser light configured for a medical procedure, the optical system transmitting a target image to a viewer; the optical system further including an optical train having at least one optical filter to remove an identified laser wavelength from the target image prior to reaching the viewer.

10. The medical telescope system of claim 9, wherein the viewer comprises a camera.

11. The medical telescope system of claim 9, wherein the at least one optical filter comprises a flat filter.

12. The medical telescope system of claim 9, wherein the at least one optical filter comprises a coated lens.

13. The medical telescope system of claim 9, wherein the telescope system further comprises a viewing apparatus and wherein the viewing apparatus includes an exterior color indicative of the identified laser wavelength filtered by the at least one optical filter.

13. The medical telescope system of claim 9, wherein the at least one optical filter has an optical density from about 3 to about 7.

14. The medical telescope system of claim 13, wherein the optical density is about 5.

15. A method of preventing overexposure of an image captured by a camera during a medical laser procedure comprising:
providing a medical telescope having an a flexible tube with an optical system mounted therein, the optical system including an integrated optical filter;
transmitting a target image through the optical system; and
filtering a selected laser wavelength with the integrated optical filter prior to transmitting the target image to a camera.

16. The method of claim 15, further comprising:
coloring a viewing apparatus on the telescope with an exterior color indicative of the selected laser wavelength filtered by the integrated optical filter.
